(19) United States
(12) Patent Application Publication
(10) Pub. No.: US 2026/0096840 A1
(43) Pub. Date: Apr. 9, 2026

(54) CABLE RETAINMENT FEATURES FOR CABLE-ACTUATED SURGICAL TOOLS

(71) Applicant: CILAG GMBH INTERNATIONAL, Zug (CH)

(72) Inventors: Austin Michael FISCHER, Cincinnati, OH (US); Christopher William BIRRI, West Chester, OH (US)

(73) Assignee: CILAG GMBH INTERNATIONAL, Zug (CH)

(21) Appl. No.: 18/906,479

(22) Filed: Oct. 4, 2024

(51) Int. Cl. A61B 18/00; A61B 34/00; A61B 34/30

(52) U.S. Cl. CPC A61B 18/00; A61B 34/30; A61B 34/71; A61B 2018/00053

(57) ABSTRACT

A surgical tool includes a drive housing, a drive input rotatably mounted to a bottom of the drive housing, and a capstan assembly arranged within the drive housing and operatively coupled to the drive input such that rotation of the drive input correspondingly actuates the capstan assembly. The capstan assembly includes a capstan that provides a cable pulley with a pulley groove sized to receive a drive cable wrapped at least partially about the capstan, a crimp pocket defined in the cable pulley and sized to receive a termination crimp secured to an end of the drive cable, and a cable retainment feature provided on the cable pulley and operable to retain the termination crimp within the crimp pocket once received therein. The termination crimp is received within the crimp pocket by forcing the termination crimp past the cable retainment feature.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

Some cable driven motion systems utilize antagonistic cable designs with multiple drive inputs to drive end effector functionality and articulation. Operation of the surgical tool can sometimes introduce slack in the drive cables, which can result in cable derailment or inadvertently feeding the drive cable into intermeshed gears.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIG. 5 is a bottom view of the drive housing of FIG. 2, according to one or more embodiments.

FIG. 6 is an exposed isometric view of the interior of the drive housing of FIG. 2, according to one or more embodiments.

FIG. 7 is an isometric side view of an example capstan assembly that may incorporate the principles of the present disclosure.

FIGS. 8A-8D depict example installation of the drive cable and the termination crimp on the cable pulley of the capstan of FIG. 7, according to one or more embodiments of the present disclosure.

FIGS. 9A-9D depict another example of the capstan, according to one or more additional embodiments of the present disclosure.

FIG. 10 is an isometric top view of the capstan, according to one or more additional embodiments.

### DETAILED DESCRIPTION

The present disclosure is related to robotic surgical systems and, more particularly, to preventing derailment and binding issues with drive cables of a cable driven surgical tool when slack accumulates in the drive cables.

Embodiments discussed herein describe a surgical tool that may include a drive housing, a drive input rotatably mounted to a bottom of the drive housing, and a capstan assembly arranged within the drive housing and operatively coupled to the drive input such that rotation of the drive input correspondingly actuates the capstan assembly. The capstan assembly may include a capstan that provides a cable pulley with a pulley groove sized to receive a drive cable wrapped at least partially about the capstan, a crimp pocket defined in the cable pulley and sized to receive a termination crimp secured to an end of the drive cable, and a cable retainment feature provided on the cable pulley and operable to retain the termination crimp within the crimp pocket once received therein. The termination crimp may be received within the crimp pocket by forcing the termination crimp past the cable retainment feature.

Accordingly, the cable retainment feature may help prevent or eliminate adverse effects of excessive or unpredictable slackening of drive cables, and potential derailment from the corresponding cable pulley. The cable retainment feature may include one or more retention bumps that, once overcome, allow the termination crimp to migrate into and be received the corresponding crimp pocket, and the retention bumps thereafter prevent the termination crimp from unseating from the crimp pocket, or otherwise shifting to an opposite side of the crimp pocket when the corresponding drive cable is slackened (i.e., during operation or assembly).

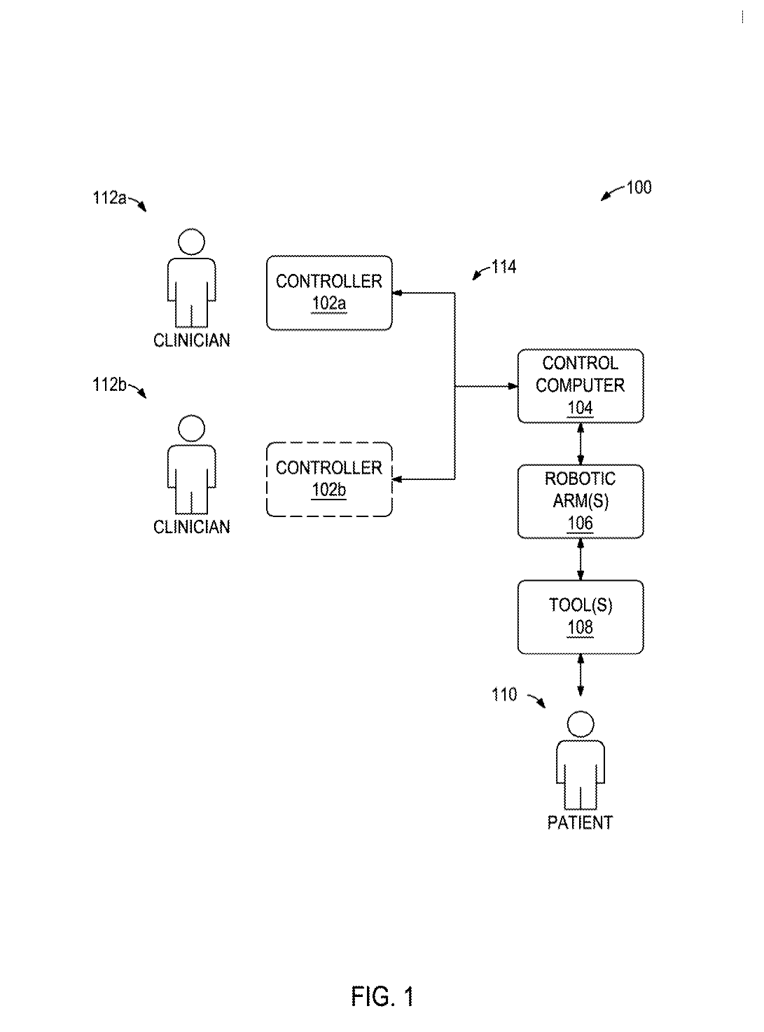

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

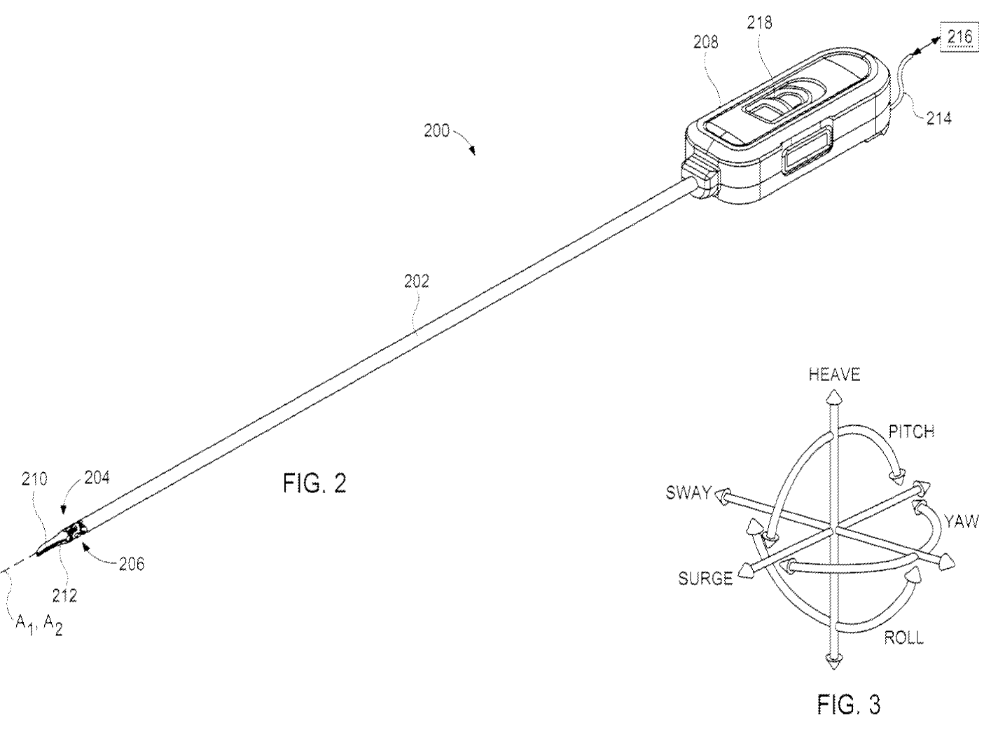

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A₁. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises a combination tissue grasper and vessel sealer that include opposing first (upper) and second (lower) jaws 210, 212 configured to move (articulate) between open and closed positions. As will be appreciated, however, the opposing jaws 210, 212 may alternatively form part of other types of end effectors such as, but not limited to, surgical scissors, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot to articulate the end effector 204 between the open and closed positions.

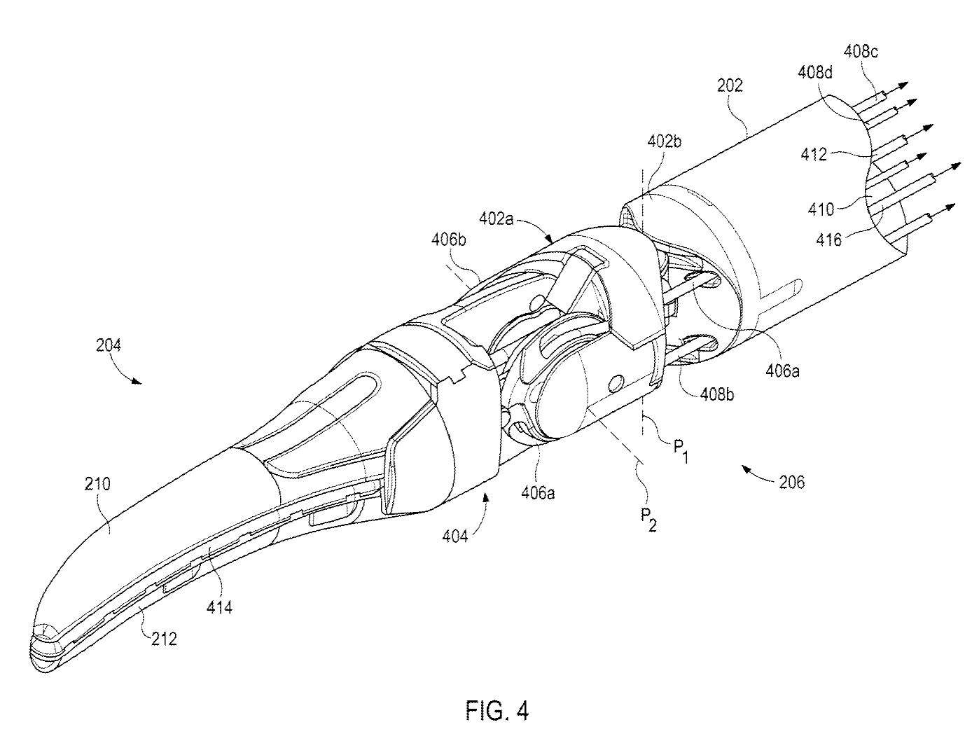

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator 216 that supplies energy, such as electrical energy (e.g., radio frequency energy), ultrasonic energy, microwave energy, heat energy, or any combination thereof, to the surgical tool 200 and, more particularly, to the end effector 204. Accordingly, the generator 216 may comprise a radio frequency (RF) source, an ultrasonic source, a direct current source, and/or any other suitable type of electrical energy source that may be activated independently or simultaneously.

In applications where the surgical tool 200 is configured for bipolar operation, the power cable 214 will include a supply conductor and a return conductor. Current can be supplied from the generator 216 to an active (or source) electrode located at the end effector 204 via the supply conductor, and current can flow back to the generator 216 via a return electrode located at the end effector 204 via the return conductor. In the case of a bipolar grasper with opposing jaws, for example, the jaws serve as the electrodes where the proximal end of the jaws are isolated from one another and the inner surface of the jaws (i.e., the area of the jaws that grasp tissue) apply the current in a controlled path through the tissue. In applications where the surgical tool 200 is configured for monopolar operation, the generator 216 transmits current through a supply conductor to an active electrode located at the end effector 204, and current is returned (dissipated) through a return electrode (e.g., a grounding pad) separately coupled to a patient's body.

The surgical tool 200 may further include a manual release switch 218 that may be manually actuated by a user (e.g., a surgeon) to override the cable driven system and thereby manually articulate or operate the end effector 204. The release switch 218 is movably positioned on the drive housing 208, and a user is able to manually move (slide) the release switch 218 from a disengaged position, as shown, to an engaged position. In the disengaged position, the surgical tool 200 is able to operate as normal. As the release switch 218 moves to the engaged position, however, various internal component parts of the drive housing 208 are simultaneously moved, thereby resulting in the jaws 210, 212 opening, which might prove beneficial for a variety of reasons. In some applications, for example, the release switch 218 may be moved in the event of an electrical disruption that renders the surgical tool 200 inoperable. In such applications, the user would be able to manually open the jaws 210, 212 and thereby release any grasped tissue and remove the surgical tool 200. In other applications, the release switch 218 may be actuated (enabled) to open the jaws 210, 212 in preparation for cleaning and/or sterilization of the surgical tool 200.

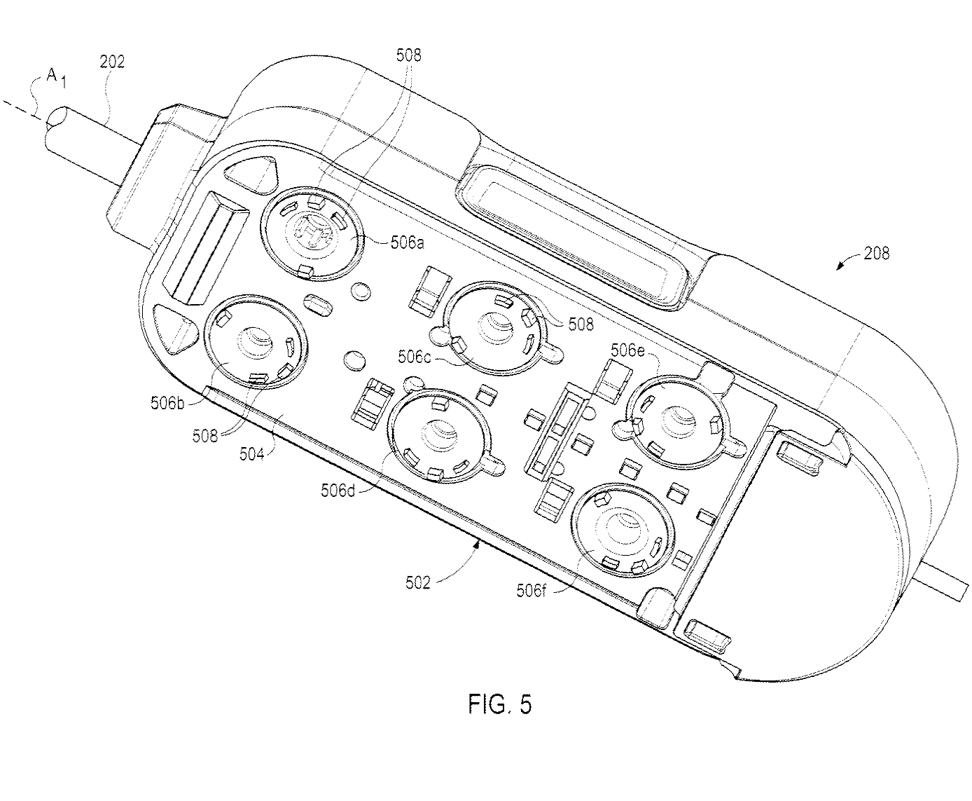

FIG. 4 is an enlarged isometric view of the distal end of the surgical tool 200. More specifically, FIG. 4 depicts an enlarged view of the end effector 204 and the wrist 206, with the jaws 210, 212 of the end effector 204 in the closed position. The wrist 206 operatively couples the end effector 204 to the shaft 202. In some embodiments, however, a shaft adapter may be directly coupled to the wrist 206 and otherwise interpose the shaft 202 and the wrist 206. Accordingly, the wrist 206 may be operatively coupled to the shaft 202 either through a direct coupling engagement where the wrist 206 is directly coupled to the distal end of the shaft 202, or an indirect coupling engagement where a shaft adapter interposes the wrist 206 and the distal end of the shaft 202. As used herein, the term “operatively couple” refers to a direct or indirect coupling engagement between two components.

To operatively couple the end effector 204 to the shaft 202, the wrist 206 includes a first or “distal” clevis 402a and a second or “proximal” clevis 402b. The clevises 402a,b are alternatively referred to as “articulation joints” of the wrist 206 and extend from the shaft 202 (or alternatively a shaft adapter). The clevises 402a,b are operatively coupled to facilitate articulation of the wrist 206 relative to the shaft 202. As illustrated, the wrist 206 also includes a linkage 404 arranged distal to the distal clevis 402a and operatively mounted to the jaws 210, 212.

The proximal end of the distal clevis 402a may be rotatably mounted or pivotably coupled to the proximal clevis 402b at a first pivot axis P₁ of the wrist 206. In some embodiments, an axle may extend through the first pivot axis P₁ and the distal and proximal clevises 402a,b may be rotatably coupled via the axle. In other embodiments, however, such as is depicted in FIG. 4, the distal and proximal clevises 402a,b may be engaged in rolling contact, such as via an intermeshed gear relationship that allows the clevises 402a,b to rotate relative to each other similar to a rolling joint.

First and second pulleys 406a and 406b may be rotatably mounted to the distal end of the distal clevis 402a at a second pivot axis P₂ of the wrist 206. The linkage 404 may be arranged distal to the second pivot axis P₂ and operatively mounted to the jaws 210, 212. The first pivot axis P₁ is substantially perpendicular (orthogonal) to the longitudinal axis A₁ of the shaft 202, and the second pivot axis P₂ is substantially perpendicular (orthogonal) to both the longitudinal axis A₁ and the first pivot axis P₁. Movement of the end effector 204 about the first pivot axis P₁ provides “yaw” articulation of the wrist 206, and movement about the second pivot axis P₂ provides “pitch” articulation of the wrist 206.

A plurality of drive cables, shown as drive cables 408a, 408b, 408c, and 408d, extend longitudinally within a lumen 410 defined by the shaft 202 (or a shaft adaptor) and extend at least partially through the wrist 206. The drive cables 408a-d may form part of the cable driven motion system housed within the drive housing 208 (FIG. 2), and may comprise cables, bands, lines, cords, wires, woven wires, ropes, strings, twisted strings, elongate members, belts, shafts, flexible shafts, drive rods, or any combination thereof. The drive cables 408a-d can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 408a-d are depicted in FIG. 4, more or less than four may be employed, without departing from the scope of the disclosure.

The drive cables 408a-d extend proximally from the end effector 204 and the wrist 206 toward the drive housing 208 (FIG. 2) where they are operatively coupled to various actuation mechanisms or devices that facilitate longitudinal movement (translation) of the drive cables 408a-d within the lumen 410. Selective actuation of the drive cables 408a-d applies tension (i.e., pull force) to the given drive cable 408a-d in the proximal direction, which urges the given drive cable 408a-d to translate longitudinally within the lumen 410.

In the illustrated embodiment, the drive cables 408a-d each extend longitudinally through the proximal clevis 402b. The distal end of each drive cable 408a-d terminates at the first or second pulleys 406a,b, thus operatively coupling each drive cable 408a-d to the end effector 204. In some embodiments, the distal ends of the first and second drive cables 408a,b may be coupled to each other and terminate at the first pulley 406a, and the distal ends of the third and fourth drive cables 408c,d may be coupled to each other and terminate at the second pulley 406b. In at least one embodiment, the distal ends of the first and second drive cables 408a,b and the distal ends of the third and fourth drive cables 408c,d may each be coupled together at corresponding ball crimps (not shown) mounted to the first and second pulleys 406a,b, respectively.

In at least one embodiment, the drive cables 408a-d may operate “antagonistically”. More specifically, when the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa. Antagonistic operation of the drive cables 408a-d can open or close the jaws 210, 212. More specifically, selective actuation of the drive cables 408a-d in known configurations or coordination will cause the jaws 210, 212 to open or close. Antagonistic operation of the drive cables 408a-d can also cause the end effector 204 to articulate at the wrist 206. More particularly, selective actuation of the drive cables 408a-d in known configurations or coordination can cause the end effector 204 to articulate about one or both of the pivot axes P₁, P₂, thus facilitating articulation of the end effector 204 in both pitch and yaw directions, either individually or simultaneously. Antagonistic operation of the drive cables 408a-d advantageously reduces the number of cables required to provide full wrist 206 motion, and also helps eliminate slack in the drive cables 408a-d, which results in more precise motion of the end effector 204.

In the illustrated embodiment, the end effector 204 is able to articulate (move) in pitch about the second or “pitch” pivot axis P₂, which is located near the distal end of the wrist 206. Thus, the jaws 210, 212 open and close in the direction of pitch. In other embodiments, however, the wrist 206 may alternatively be configured such that the second pivot axis P₂ facilitates yaw articulation of the jaws 210, 212, without departing from the scope of the disclosure.

In some embodiments, an electrical conductor 412 may also extend longitudinally within the lumen 410, through the wrist 206, and terminate at an electrode 414 to supply electrical energy to the end effector 204. In some embodiments, the electrical conductor 412 may comprise a wire, but may alternatively comprise a rigid or semi-rigid shaft, rod, or strip (ribbon) made of a conductive material. The electrical conductor 412 may be entirely or partially covered with an insulative covering (overmold) made of a non-conductive material. Using the electrical conductor 412 and the electrode 414, the end effector 204 may be configured for monopolar or bipolar RF operation.

In the illustrated embodiment, the end effector 204 comprises a combination tissue grasper and vessel sealer that includes a knife (not visible), alternately referred to as a “cutting element” or “blade.” The knife is aligned with and configured to traverse a guide track (not visible) defined longitudinally in one or both of the upper and lower jaws 210, 212. The knife may be operatively coupled to the distal end of a drive rod 416 that extends longitudinally within the lumen 410 and passes through the wrist 206. Longitudinal movement (translation) of the drive rod 416 correspondingly moves the knife within the guide track(s). Similar to the drive cables 408a-d, the drive rod 416 may form part of the actuation systems housed within the drive housing 208 (FIG. 2). Selective actuation of a corresponding drive input will cause the drive rod 416 to move distally or proximally within the lumen 410, and correspondingly move the knife 416 in the same longitudinal direction.

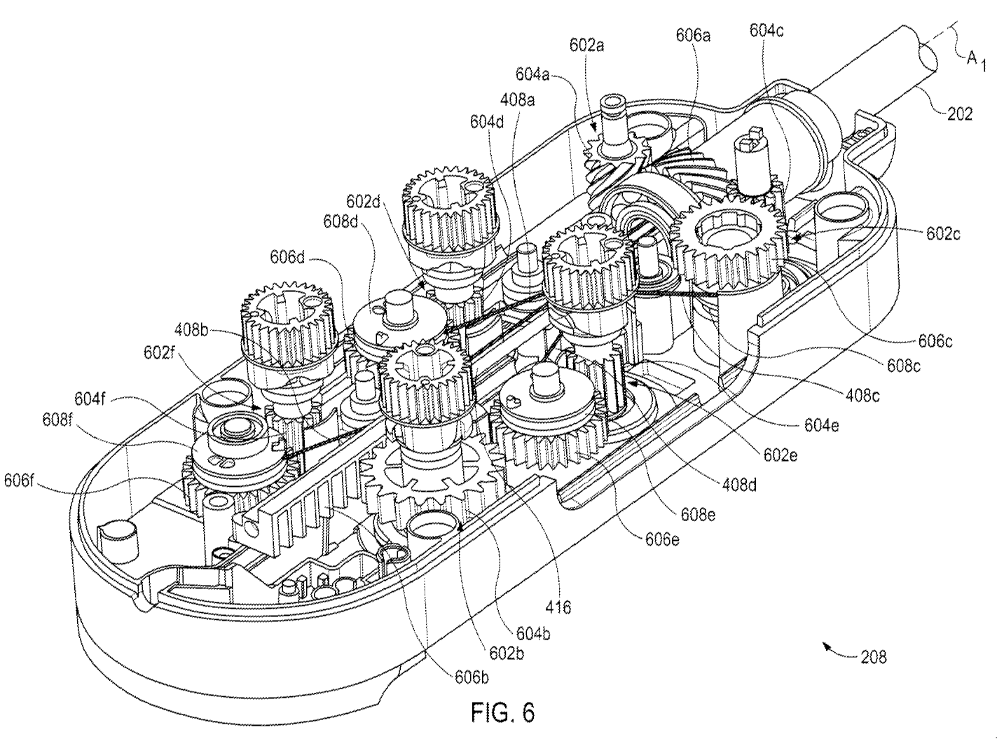

FIG. 5 is a bottom view of the drive housing 208, according to one or more embodiments. As illustrated, the drive housing 208 may include a tool mounting portion 502 used to operatively couple the drive housing 208 to a tool driver of a robotic manipulator. The tool mounting portion 502 may releasably couple the drive housing 208 to a tool driver in a variety of ways, such as by clamping thereto, clipping thereto, or slidably mating therewith. In some embodiments, the tool mounting portion 502 may include an array of electrical connecting pins, which may be coupled to an electrical connection on the mounting surface of the tool driver. While the tool mounting portion 502 is described herein with reference to mechanical, electrical, and magnetic coupling elements, it should be understood that a wide variety of telemetry modalities might be used, including infrared, inductive coupling, or the like.

The tool mounting portion 502 includes and otherwise provides an interface 504 configured to mechanically, magnetically, and/or electrically couple the drive housing 208 to the tool driver. As illustrated, the interface 504 includes and supports a plurality of drive inputs, shown as drive inputs 506a, 506b, 506c, 506d, 506e, and 506f. Each drive input 506a-f comprises a rotatable disc configured to align with and couple to a corresponding actuator or “drive output” of a tool driver, such that rotation (actuation) of a given drive output drives (rotates) a corresponding one of the drive inputs 506a-f. Each drive input 506a-f may provide or define one or more surface features 508 configured to align with mating surface features provided on the corresponding drive output. The surface features 508 can include, for example, various protrusions and/or indentations that facilitate a mating engagement. In some embodiments, some or all of the drive inputs 506a-f may include one surface feature 508 that is positioned closer to an axis of rotation of the associated drive input 506a-f than the other surface feature(s) 508. This may help to ensure positive angular alignment of each drive input 506a-f.

In some embodiments, actuation of the first drive input 506a may be configured to control rotation of the shaft 202 about its longitudinal axis A₁. The shaft 202 may be rotated clockwise or counter-clockwise depending on the rotational actuation of the first drive input 506a. In some embodiments, actuation of the second, third, fourth, and fifth drive inputs 506b-e may be configured to operate movement (axial translation) of the drive cables 408a-d (FIG. 4), which results in the actuation of the wrist 106 (FIG. 4) and/or articulation (operation) of the end effector 204 (FIG. 4). In some embodiments, actuation of the sixth drive input 506f may be configured to advance and retract the drive rod 416 (FIG. 4), and thereby correspondingly advance or retract the knife at the end effector 204. Each of the drive inputs 506a-f may be actuated based on user inputs communicated to the tool driver coupled to the interface 504, and the user inputs may be received via a computer system incorporated into the robotic surgical system.

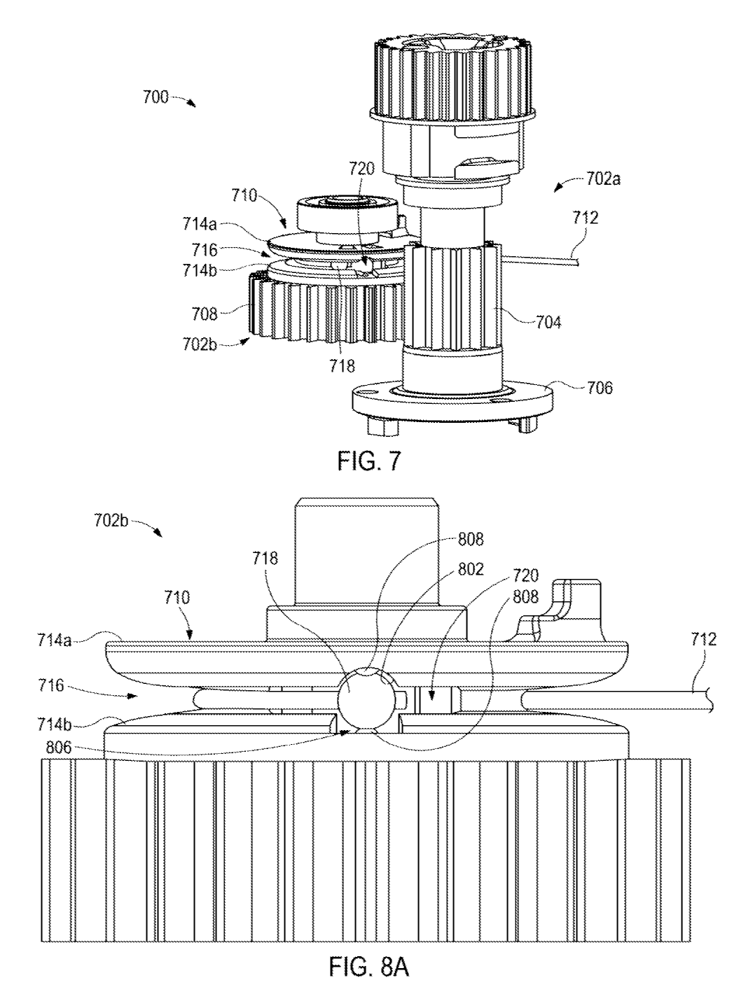

FIG. 6 is an exposed isometric view of the interior of the drive housing 208. Several component parts that may be otherwise contained within the drive housing 208 are not shown in FIG. 6 to enable discussion of the depicted component parts. As illustrated, the drive housing 208 houses and otherwise contains a plurality of capstan assemblies operable to operate surgical tool 200 (FIG. 2). In particular, a first capstan assembly 602a is contained (housed) within the drive housing 208. As illustrated, the first capstan assembly 602a may include a drive gear 604a, which may be operatively coupled to or extend from the first drive input 506a (FIG. 5) such that actuation of the first drive input 506a results in rotation of the drive gear 604a. In the illustrated embodiment, the drive gear 604a comprises a worm gear, which may be configured to mesh and interact with a driven gear 606a secured within the drive housing 208 and operatively coupled to the shaft 202 such that rotation of the driven gear 606a correspondingly rotates the shaft 202. Accordingly, actuation of the first capstan assembly 602a, via actuation of the first drive input 506a, will drive the driven gear 606a and thereby control rotation of the elongate shaft 202 about the longitudinal axis A₁.

The drive housing 208 may further contain or house a second capstan assembly 602b, which may include a drive gear 604b operatively coupled to or extending from the sixth drive input 506f (FIG. 5) such that actuation of the sixth drive input 506f results in rotation of the drive gear 604b. The drive gear 604b is arranged to intermesh with a driven gear 606b positioned within the drive housing 208. In the illustrated embodiment, the driven gear 606b comprises a rack gear longitudinally translatable within the drive housing 208 as acted upon by the drive gear 604b. The drive rod 416 may be operatively coupled to the driven gear 606b and extend distally therefrom to the end effector 204 (FIGS. 2 and 4). Accordingly, actuation of the second capstan assembly 602b, via actuation of the sixth drive input 506f, will cause the driven gear 606b to longitudinally translate and correspondingly advance or retract the drive rod 416 and the knife coupled to the end of the drive rod 416 at the end effector 204.

The drive housing 208 further contains or houses third, fourth, fifth, and sixth capstan assemblies 602c, 602d, 602e, and 602f, alternately be referred to as “drive cable” capstan assemblies since they are operable to actuate the drive cables 408a-d, as described below. While four “drive cable” capstan assemblies 602c-f are depicted in FIG. 6, alternative embodiments may include more or less than four, depending on how many drive cables 408a-d are used.

In the illustrated embodiment, the third capstan assembly 602c is actuated through operation (rotation) of the second drive input 506b (FIG. 5), the fourth capstan assembly 602d is actuated through operation (rotation) of the third drive input 506c (FIG. 5), the fifth capstan assembly 602e is actuated through operation (rotation) of the fourth drive input 506d (FIG. 5), and the sixth capstan assembly 602f is actuated through operation (rotation) of the fifth drive input 506e (FIG. 5). As illustrated, each capstan assembly 602c-f includes a drive gear 604c, 604d, 604e, and 604f that is coupled to or extends from the corresponding drive input 506b-e, respectively, such that actuation (rotation) of the drive input 506b-e correspondingly rotates the associated drive gear 604c-f, respectively.

Moreover, each drive gear 604c-f is positioned to mesh and interact with a corresponding driven gear 606c, 606d, 606e, and 606f rotatably mounted within the drive housing 208. Each driven gear 606c-f includes or is otherwise coupled to a corresponding cable pulley 608c, 608d, 608e, and 608f, and each cable pulley 608c-f is configured to be operatively coupled to (e.g., has wrapped there around, at least partially) a corresponding one of the drive cables 408a-d. In the illustrated embodiment, the first drive cable 408a terminates at cable pulley 608d ultimately driven by actuation of the fourth capstan assembly 602d, the second drive cable 408b terminates at cable pulley 608f ultimately driven by actuation of the sixth capstan assembly 602f, the third drive cable 408c terminates at cable pulley 608c ultimately driven by actuation of the third capstan assembly 602c, and the fourth drive cable 408d terminates at cable pulley 608e ultimately driven by actuation of the fifth capstan assembly 602e.

Accordingly, actuation of the fourth capstan assembly 602d (via operation of the third drive input 506c of FIG. 5) will correspondingly control movement of the first drive cable 408a; actuation of the sixth capstan assembly 602f (via operation of the fifth drive input 506e of FIG. 5) will correspondingly control movement of the second drive cable 408b; actuation of the third capstan assembly 602c (via operation of the second drive input 506b of FIG. 5) will correspondingly control movement of the third drive cable 408c; and actuation of the fifth capstan assembly 602e (via operation of the fourth drive input 506d of FIG. 5) will correspondingly control movement of the fourth drive cable 408d.

### Capstan Cable Crimp Retainment Features

Still referring to FIG. 6, an antagonistic architecture for the drive cables 408a-d enables the amount of tension in each drive cable 408a-d to be selectively changed, which helps accurately control operation and actuation of the end effector 204 (FIGS. 2 and 4). Antagonistic architecture, however, has the drawback of potential accumulation of slack in a single or multiple cables 408a-d during operation. For example, if a given drive input 506b-e (FIG. 5) is driven excessively in the slack direction, there is a risk that slack may accumulate in the corresponding drive cable 408a-d, which may result in the drive cable 408a-d derailing from the corresponding cable pulley 608c-f. If the drive cable 408a-d derails, the surgical tool 200 (FIG. 2) effectively becomes inoperable. Moreover, besides risking the accumulation of slack, the drive cable 408a-d could also fully unwrap from the corresponding cable pulley 608c-f. In such a scenario, the termination crimp (e.g., a ball crimp) that attaches the drive cable 408a-d to the corresponding capstan assembly 602d-f could potentially release from the cable pulley 608d-f, which would also result in device failure.

According to embodiments of the present disclosure, the adverse effects of excessive or unpredictable slackening of the drive cables 408a-d, and potential derailment from the corresponding cable pulleys 608c-f, may be mitigated and otherwise prevented by including a cable retainment feature on the cable pulleys 608c-f. As described herein, the cable retainment feature may be arranged within the pulley groove, which is contiguous with a crimp pocket sized to receive the termination crimp. The cable retainment feature may include one or more retention bumps that, once overcome, allow the termination crimp to migrate into and be received within a corresponding crimp pocket. The retention bumps thereafter prevent the termination crimp from unseating from the crimp pocket, or otherwise shifting to an opposite side of the crimp pocket when the corresponding drive cable is slackened (i.e., during operation or assembly), thus helping to prevent cable derailment during operation. In some embodiments, the crimp pocket may comprise an elongate, arcuate cavity, and the termination crimp can be received within either angular end of the crimp pocket. This allows the cable retainment feature to be used at multiple device locations where the drive cable can wrap in either direction.

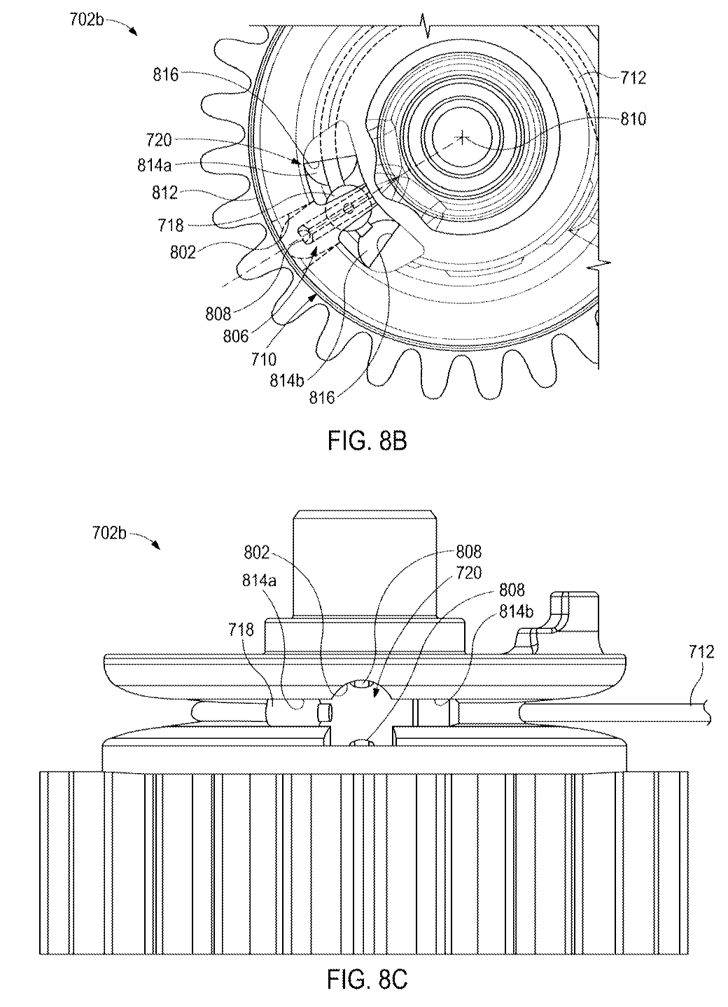

FIG. 7 is an isometric side view of an example capstan assembly 700 that may incorporate the principles of the present disclosure. The capstan assembly 700 may be the same as or similar to any of the capstan assemblies 602c-f as described above with reference to FIG. 6. Accordingly, the capstan assembly 700 may be arranged within the drive housing 208 (FIG. 6) and thereby form part of the surgical tool 200 (FIG. 2).

In some embodiments, as illustrated, the capstan assembly 700 may include a drive capstan 702a and a driven capstan 702b arranged to be driven by the drive capstan 702a. More specifically, the drive capstan 702a includes a drive gear 704 that is coupled to or extends from a corresponding drive input 706, and actuation (rotation) of the drive input 706 correspondingly rotates the drive gear 704. The drive input 706 may be the same as or similar to any of the drive inputs 506b-e (FIG. 5). The drive gear 704 is positioned to mesh and interact with a driven gear 708 of the driven capstan 702b, such that actuation (rotation) of the drive capstan 702a correspondingly drives (rotates) the driven capstan 702b.

In other embodiments, however, the capstan assembly 700 may only include the driven capstan 702b, which may be operatively coupled to or otherwise extend from the drive input 706. In such embodiments, actuation (rotation) of the drive input 706 will correspondingly and directly rotate the driven capstan 702b, and the drive capstan 702a may therefore be omitted. Moreover, in such embodiments, the driven capstan 702b may alternately be referred to as the “drive capstan” or simply the “capstan”. Accordingly, for purposes of the discussion, the driven capstan 702b will be referred to herein as simply “the capstan 702b”.

As illustrated, the capstan 702b includes a cable pulley 710 configured to receive and secure a drive cable 712 thereto. The cable pulley 710 may be the same as or similar to any of the cable pulleys 608c-f of FIG. 6, and the drive cable 712 may be the same as or similar to any of the drive cables 408a-d of FIGS. 4 and 6. The cable pulley 710 may provide upper and lower pulley flanges 714a and 714b, and a pulley groove 716 may be defined by or between the pulley flanges 714a,b and sized to receive the drive cable 712. The drive cable 712 is wrapped at least partially about the capstan 702b within the pulley groove 716.

As illustrated, a termination crimp 718 may be secured to an end of the drive cable 712 to help retain the drive cable 712 within the pulley groove 716 and otherwise operatively coupled to the capstan 702b. The termination crimp 718, alternately referred to as a “termination boss” or “termination fitting,” may comprise any device or structure, or exhibit any geometry capable of helping to retain the drive cable 712 within the pulley groove 716. In the illustrated embodiment, the termination crimp 718 comprises a ball crimp, which exhibits a generally spherical shape and circular cross-section. In other embodiments, however, the termination crimp 718 may comprise a barrel crimp, a puck-shaped crimp, a truncated cone, a rectangular prism (or similar with more or less sides), a ball and shank, a barrel with flange, or any combination thereof, without departing from the scope of the disclosure. The various types of the termination crimp 718 may be attached to the end of the drive cable 712 via a variety of processes including, but not limited to, crimping, welding, overmolding, peening, application of an adhesive, riveting, threading, or any combination thereof.

The drive cable 712 is installed on the cable pulley 710 by wrapping the drive cable 712 at least partially about the capstan 702b within the pulley groove 716, and receiving the termination crimp 718 within a crimp pocket 720 defined within the cable pulley 710. Once the termination crimp 718 is properly received within the crimp pocket 720, the capstan 702b may be operated. In example operation of the capstan 702b, for instance, the drive cable 712 is placed in tension, and actuation (rotation) of the capstan 702b correspondingly causes the drive cable 712 to move longitudinally (e.g., within the shaft 202 of FIG. 2) relative to the drive housing 208 (FIGS. 2 and 6). Accordingly, torque applied to the capstan 702b is transferred from the capstan 702b to the drive cable 712.

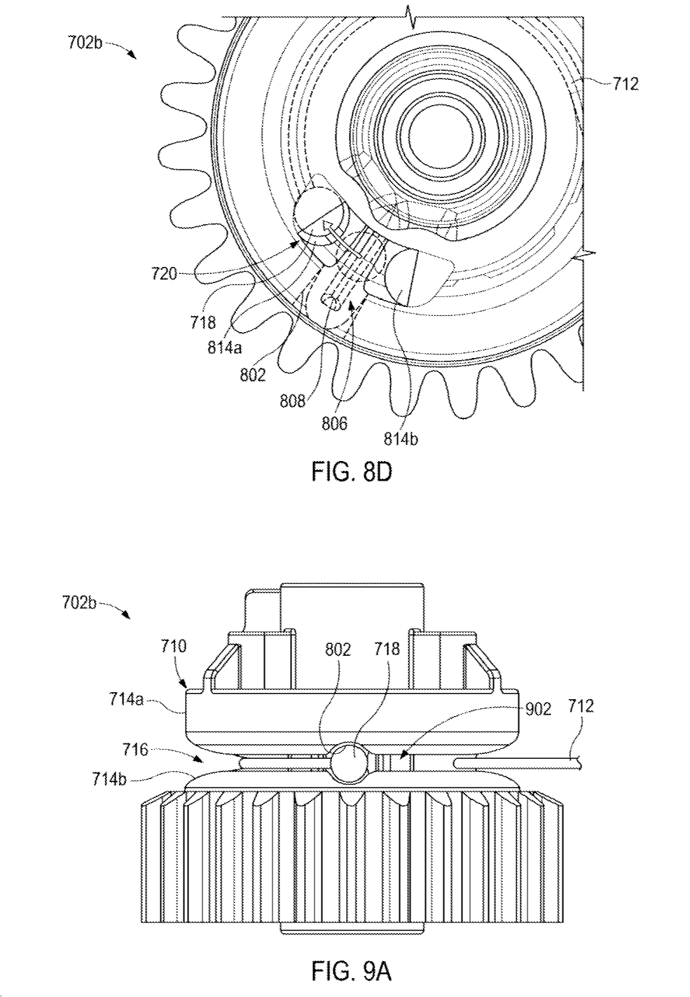

FIGS. 8A-8D depict example installation of the drive cable 712 and the termination crimp 718 on the capstan 702b, according to one or more embodiments of the present disclosure. FIGS. 8A and 8C depict side views of the capstan 702b, and FIGS. 8B and 8D are top views of the capstan 702b with various structural elements depicted in phantom (dashed lines) to enable viewing of the position (location) of the drive cable 712 and the termination crimp 718. FIGS. 8A-8B depict the termination crimp 718 in a first or “unseated state”, where the termination crimp 718 is located either outside of the crimp pocket 720 or otherwise in the process of being received therein, and FIGS. 8C and 8D depict the termination crimp 718 in a second or “seated state”, where the termination crimp 718 is successfully received within the crimp pocket 720.

Referring first to FIGS. 8A-8B, as illustrated, the cable pulley 710 may provide or otherwise define a crimp or “termination” aperture 802 sized to receive the termination crimp 718 (e.g., a ball crimp), and guide the termination crimp 718 into the crimp pocket 720 defined within the cable pulley 710. Accordingly, the termination aperture 802 may be contiguous with and lead into the crimp pocket 720 such that the termination crimp 718 is received within the crimp pocket 720 by first passing through the termination aperture 802. As will be appreciated, the size and geometry of the termination aperture 802 may be modified to receive the corresponding size and geometry of the termination crimp 718. In some embodiments, for example, the termination crimp 718 may exhibit a generally spherical cross-sectional shape. In such embodiments, the termination aperture 802 may at least partially exhibit a circular cross-sectional shape larger than the termination crimp 718 and thereby sized to receive the spherical termination crimp 718.

In some embodiments, as best seen in FIG. 8A, the termination aperture 802 may be cooperatively defined by the upper and lower pulley flanges 714a,b. In such embodiments, portions of the termination aperture 802 may be defined by each pulley flange 714a,b to accommodate the size and geometry of the termination crimp 718. In other embodiments, however, the termination aperture 802 may be defined by only one of the pulley flanges 714a,b.

Similar to the termination aperture 802, in some embodiments, the crimp pocket 720 may be cooperatively defined by the upper and lower pulley flanges 714a,b. In other embodiments, however, the crimp pocket 720 may be defined by only one of the pulley flanges 714a,b, without departing from the scope of the disclosure.

As briefly mentioned above, the capstan 702b may further include a cable retainment feature 806 that may help retain the termination crimp 718 within the crimp pocket 720. In the illustrated embodiment, the cable retainment feature 806 includes one or more retention bumps 808 arranged within the pulley groove 716 at the termination aperture 802, such as at the lead-in into the termination aperture 802. In the illustrated embodiment, the cable retainment feature 806 includes two retention bumps 808, where one retention bump 808 is provided on the upper pulley flange 714a and the second retention bump 808 is provided on the lower pulley flange 714b. In some embodiments, as illustrated, the retention bumps 808 comprise elongate protrusions vertically offset from each other on opposing upper and lower portions of the termination aperture 802.

As best seen in FIG. 8B, the retention bumps 808 may extend substantially perpendicular to a rotational axis 810 of the capstan 702b. Moreover, in some embodiments, one or both of the retention bumps 808 may extend transversely through the crimp pocket 720 and at least partially into the termination aperture 802. In at least one embodiment, however, the retention bumps 808 may terminate prior to reaching the outer radial circumference 812 of the cable pulley 710.

In some embodiments, to receive the termination crimp 718 within the crimp pocket 720, the termination crimp 718 must first bypass the retention bumps 808. In such embodiments, the retention bumps 808 may facilitate an interference fit that must be physically (manually) overcome to enable the termination crimp 718 to enter the crimp pocket 720. Accordingly, in such embodiments, the termination crimp 718 may be manually forced against and past the retention bumps 808. Forcing the termination crimp 718 past the retention bumps 808 may cause one or both of the pulley flanges 714a,b to flex outward, or may alternatively (or in addition thereto) elastically or partially deform the retention bumps 808, and thereby allow the termination crimp 7182 bypass the retention bumps 808. In such embodiments, the cable pulley 710 (or at least the pulley flanges 714a,b) may be made of a flexible material, such as a polymer.

Still referring to FIG. 8B, in some embodiments, the crimp pocket 720 may comprise an arcuate cavity providing first and second pocket chambers 814a and 814b separated by the retention bumps 808. In at least one embodiment, the crimp pocket 720 may be symmetric, and the first and second pocket chambers 814a,b may be the same size to enable the termination crimp 718 to be received within either pocket chamber 814a,b. Accordingly, the retention bumps 808 may angularly interpose the pocket chambers 814a,b. In other embodiments, however, the crimp pocket 720 may only include a single pocket chamber, without departing from the scope of the disclosure.

When installing the drive cable 712 on the cable pulley 710, the termination crimp 718 is forced past the retention bumps 808 and selectively received within one of the pocket chambers 814a,b. The particular pocket chamber 814a,b selected to receive the termination crimp 718 will depend on which direction the drive cable 712 is wrapped about the cable pulley 710. In the illustrated embodiment, for example, since the drive cable 712 is wrapped about the cable pulley 710 clockwise, the termination crimp 718 will be received within the first pocket chamber 814a. In contrast, if the drive cable 712 was to be wrapped about the cable pulley 710 counterclockwise, the termination crimp would eventually be received within the second pocket chamber 814b. The symmetric design of the crimp pocket 720 allows the capstan 702b to be arranged in various locations and configuration within the drive housing 208 (FIGS. 2 and 6).

Accordingly, each pocket chamber 814a,b may be sized to receive and seat the termination crimp 718. Still referring to FIG. 8B, in some embodiments, one or both of the pocket chambers 814a,b may provide or otherwise define a pocket window 816 through which portions of the drive cable 712 and/or the termination crimp 718 may be visible when the termination crimp 718 is received within the particular pocket chamber 814a,b. Accordingly, the pocket windows 816 may enable visual confirmation of proper seating of the termination crimp 718, but may also be useful in helping to facilitate physical inspection techniques, such as gaging to confirm appropriate seating within the pocket chamber 814a,b, or for properly clocking of the capstan 702. Each pocket window 816 may exhibit a size or geometry that prevents the termination crimp 718 from extending fully therethrough and thereby escaping the crimp pocket 720. Moreover, the pocket windows 816 may enable forming of the corresponding pocket chamber 814a,b through molding.

As noted above, FIGS. 8A-8B depict the termination crimp 718 in the first or “unseated state”, where the termination crimp 718 is located either outside of the crimp pocket 720 or otherwise in the process of being received within the crimp pocket 720 and, more particularly, within one of the pocket chambers 814a,b. In contrast, FIGS. 8C-8D depict the termination crimp 718 in the second or “seated state”, where the termination crimp 718 is successfully received within the one of the pocket chambers 814a,b of the crimp pocket 720.

Referring to FIGS. 8C-8D, the termination crimp 718 is depicted as having moved from the unseated state to the seated state and is thereby received within the first pocket chamber 814a of the crimp pocket 720. After being received within the first pocket chamber 814a, the retention bumps 808 prevent the termination crimp 718 from transitioning either out of the crimp pocket 720 via the termination aperture 802, or to the opposite side of the crimp pocket 720 and into the second pocket chamber 814b. Accordingly, not only does the cable retainment feature 806 (e.g., the retention bumps 808) facilitate an interference fit for installing the termination crimp 718, the cable retainment feature 806 also helps maintain the termination crimp 718 within the crimp pocket 704, and thereby helps to prevent inadvertent derailment of the drive cable 712 from the cable pulley 710 during operation and/or assembly.

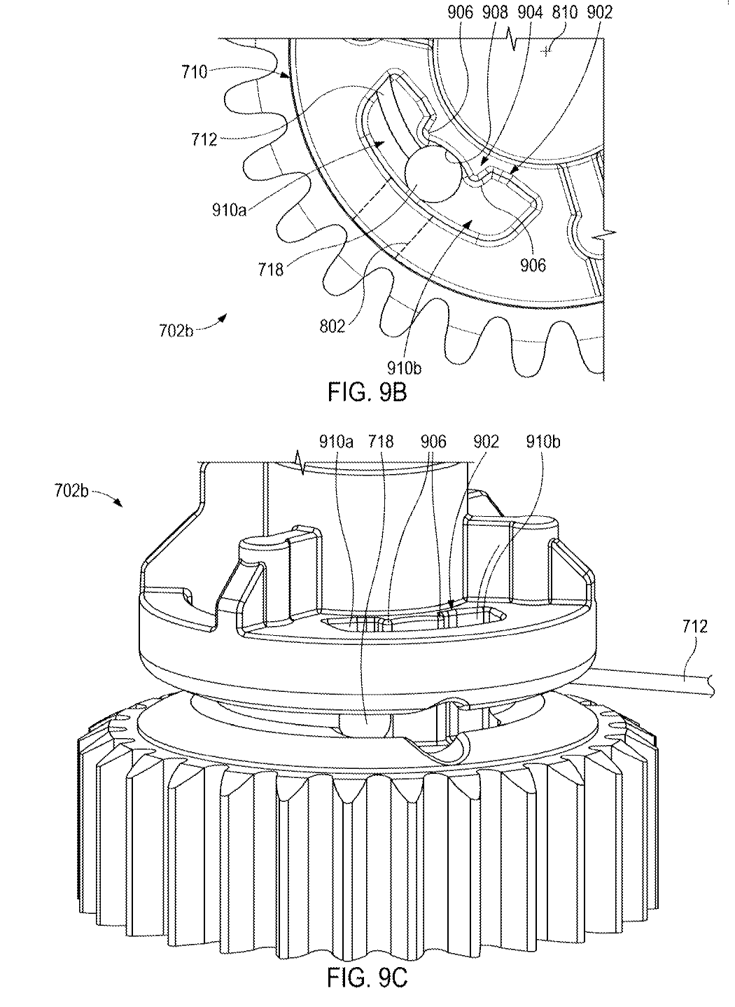

FIGS. 9A-9D depict another example of the capstan 702b, according to one or more additional embodiments of the present disclosure. FIGS. 9A and 9C depict side views of the capstan 702b, and FIGS. 9B and 9D are top views of the capstan 702b. Moreover, FIGS. 9A-9B depict the termination crimp 718 in the first or “unseated state”, and FIGS. 9C-9D depict the termination crimp 718 and the second or “seated state”.

Referring first to FIGS. 9A-9B, as illustrated, the cable pulley 710 provides the termination aperture 802 sized to receive the termination crimp 718 and guide the termination crimp 718 into a crimp pocket 902 defined within the cable pulley 710. The termination aperture 802 may be contiguous with and lead into the crimp pocket 902 such that the termination crimp 718 may be received within the crimp pocket 902 by first passing through the termination aperture 802. In some embodiments, as illustrated, the crimp pocket 902 may be cooperatively defined by the upper and lower pulley flanges 714a,b (FIG. 9A). In other embodiments, however, the crimp pocket 902 may be defined by only one of the pulley flanges 714a,b, without departing from the scope of the disclosure.

As best seen in FIG. 9B, the capstan 702b may further include a cable retainment feature 904 to help retain the termination crimp 718 within the crimp pocket 902. In the illustrated embodiment, the cable retainment feature 904 is provided within the pulley groove 716 (FIG. 9A) at the end of the termination aperture 802 and closest to the rotational axis 810 of the capstan 702b. More specifically, the cable retainment feature 904 may be provided within the crimp pocket 902. As illustrated, the cable retainment feature 904 includes a pair of retention bumps 906 angularly separated by an arcuate surface 908.

To receive and seat the termination crimp 718 within the crimp pocket 902, the termination crimp 718 is first introduced into the termination aperture 802 and advanced toward the crimp pocket 902 until engaging the cable retainment feature 904 and, more particularly, the arcuate surface 908. Similar to the termination aperture 802 of FIGS. 8A-8D, the crimp pocket 902 may comprise an arcuate cavity providing first and second pocket chambers 910a and 910b that reside on opposing sides of the cable retainment feature 904 and, more particularly, on opposing sides of the retention bumps 906. In other embodiments, however, the crimp pocket 902 may only have one pocket chamber. In embodiments with two pocket chambers 910a,b, the crimp pocket 720 may be symmetric such that the pocket chambers 910a,b may be the same size to enable the termination crimp 718 to be received within either pocket chamber 910a,b. To fully receive the termination crimp 718 within the crimp pocket 902, the termination crimp 718 is selectively forced past one of the retention bumps 906 and thereafter received within the adjacent pocket chamber 910a,b. The material of at least the cable retainment feature 904 (and possibly the entire capstan 702b) may be flexible and otherwise allow a small amount of flexure to enable the termination crimp 718 to bypass the retention bumps 906 with the proper amount of force. The particular pocket chamber 910a,b selected to receive the termination crimp 718 will depend on which direction the drive cable 712 is wrapped about the cable pulley 710, as generally described above.

As noted above, FIGS. 9A-9B depict the termination crimp 718 in the first or “unseated state”, where the termination crimp 718 is located either outside of the crimp pocket 902 or otherwise in the process of being received within the crimp pocket 902 and, more particularly, within one of the pocket chambers 910a,b. In contrast, FIGS. 9C-9D depict the termination crimp 718 in the second or “seated state”, where the termination crimp 718 is successfully received within the one of the pocket chambers 910a,b of the crimp pocket 902.

Referring to FIGS. 9C-9D, the termination crimp 718 is depicted as having moved from the unseated state to the seated state, and is thereby received within the first pocket chamber 910a of the crimp pocket 902. After being received within the first pocket chamber 910a, the retention bump 906 adjacent the first pocket chamber 910a prevents the termination crimp 718 from transitioning either out of the crimp pocket 902 via the termination aperture 802, or to the opposite side of the crimp pocket 902 and into the second pocket chamber 910b. Accordingly, the cable retainment feature 904 (e.g., the retention bumps 906) facilitates an interference fit for installing the termination crimp 718, and also helps maintain the termination crimp 718 within the crimp pocket 704, and thereby prevent inadvertent derailment of the drive cable 712 during operation and/or assembly.

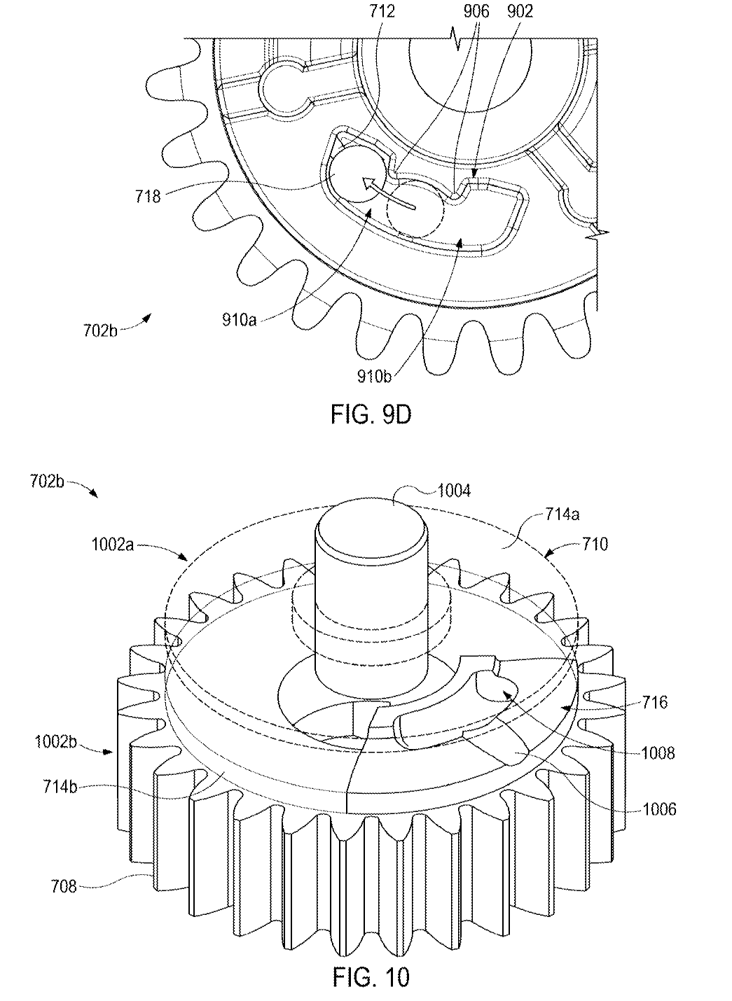

FIG. 10 is an isometric top view of the capstan 702b, according to one or more additional embodiments. In the illustrated embodiment, the capstan 702b may include and otherwise be made up of two or more component parts, shown as first and second component parts 1002a and 1002b. The first component part 1002a is shown in phantom (dashed lines) to enable viewing of the internal features of the capstan 702b.

As illustrated, the first component part 1002a may comprise the upper pulley flange 714a, and the lower component part 1002b may comprise a combination of the lower pulley flange 714b and the driven gear 708. In some embodiments, the second component part 1002b may further include a bearing post 1004, and the first component part 1002a may be configured to be received about the bearing post 1004.

Each component part 1002a,b may be individually manufactured and subsequently joined to form the capstan 702b. Moreover, joining the component parts 1002a,b will also form the cable pulley 710 and otherwise define the pulley groove 716, as generally described above. The component parts 1002a,b may be joined via a variety of coupling processes including, but not limited to, welding, an adhesive, a mechanical attachment (e.g., pinning, etc.), overmolding, peening, crimping, or any combination thereof.

In some embodiments, the capstan 702b may also provide or otherwise define a termination aperture 1006 that leads into a crimp pocket 1008 defined within the cable pulley 710. In other embodiments, however, the termination aperture 1006 may be omitted. More specifically, the termination crimp 718 (FIGS. 8A-8D, 9A-9D) can initially be received within the crimp pocket 1008 prior to joining the component parts 1002a,b. In such applications, there would be no need to include the termination aperture 1006, which would otherwise allow the termination crimp 718 to be advanced into the crimp pocket 1008.

In some embodiments, as illustrated, the crimp pocket 1008 may be cooperatively defined by the first and second component parts 1002a,b. In other embodiments, however, the crimp pocket 1008 may be defined by only one of the component parts 1002a,b, without departing from the scope of the disclosure. Moreover, in some embodiments, the crimp pocket 1008 may comprise an arcuate cavity, and the termination crimp 718 (FIGS. 8A-8D, 9A-9D) may be movable between the opposing angular ends of the crimp pocket 1008. In other embodiments, however, the crimp pocket 1008 may be sized and exhibit a geometry to receive and simultaneously secure the termination crimp 718. For example, in embodiments where the termination crimp 718 comprises a ball crimp, the crimp pocket 1008 may exhibit a spherical geometry sized to receive and securely seat the ball crimp. In such embodiments, the termination crimp 718 may be received within the crimp pocket 1008 before the first and second component parts 1002a,b are joined, and the termination crimp 718 will be captured between the component parts 1002a,b upon joining. Moreover, in such embodiments, this enables a full spherical interface at the termination crimp 718, thus removing the possibility of play, which could result in derailment. A spherical pocket interface creates a larger surface area of contact to the termination feature, thereby reducing stress and deformation of the crimp pocket 1008.

In some embodiments, the capstan 702b may include either of the cable retainment features 806, 902 described herein with reference to FIGS. 8A-8D, 9A-9D, respectively. In other embodiments, however, the cable retainment feature 806, 902 may be omitted since the termination crimp 718 (FIGS. 8A-8D, 9A-9D) may be initially received into the crimp pocket 1008 prior to joining the component parts 1002a,b, and there would therefore be no need to force the termination crimp 718 past any retention bumps or the like. In such embodiments, the first and second component parts 1002a,b may be made of a rigid material, such as a metal (e.g., stainless steel), which would make the cable pulley 710 more robust. In other embodiments, however, only one of the component parts 1002a,b may be made of a rigid material, while the other component part 1002a,b may be made of a flexible material, such as a polymer. In yet other embodiments, the first and second component parts 1002a,b may each be made of a flexible material, without departing from the scope of the disclosure.

While the cable retainment features 806, 904 described herein include retention bumps 808, 906 that are defined at the termination aperture 802 (FIGS. 8A-8D) or as forming part of the crimp pocket 902 (FIGS. 9A-9D), respectively, other cable retainment features are also contemplated herein. For example, an alternative cable retainment feature could include one or more retention bumps formed on opposing underside (facing) surfaces of the pulley flanges 7141a,b. Portions of the drive cable 712 can be forced past the retention bumps, and thereby retain the drive cable 712 via an interference fit within the pulley groove 716. In this embodiment, the drive cable 712 would be pulled past the retention bumps at an area of the drive cable 712 that has less tendency to derail from the pulley groove 716. Accordingly, these retention bumps for retaining the drive cable 712 within the pulley groove 716 can be used independently or, for extra security, in combination with the retention bumps 808, 906 described herein to help retain the termination crimp 718.

### Embodiments disclosed herein include:

A. A surgical tool includes a drive housing, a drive input rotatably mounted to a bottom of the drive housing, and a capstan assembly arranged within the drive housing and operatively coupled to the drive input such that rotation of the drive input correspondingly actuates the capstan assembly, the capstan assembly including a capstan that provides a cable pulley with a pulley groove sized to receive a drive cable wrapped at least partially about the capstan, a crimp pocket defined in the cable pulley and sized to receive a termination crimp secured to an end of the drive cable, and a cable retainment feature provided on the cable pulley and operable to retain the termination crimp within the crimp pocket once received therein, wherein the termination crimp is received within the crimp pocket by forcing the termination crimp past the cable retainment feature.

B. A capstan assembly for a surgical tool includes a capstan arrangeable within a drive housing of the surgical tool and operatively couplable to a drive input rotatably mounted to a bottom of the drive housing such that rotation of the drive input correspondingly actuates the capstan, the capstan including a cable pulley with a pulley groove sized to receive a drive cable wrapped at least partially about the capstan, a crimp pocket defined in the cable pulley and sized to receive a termination crimp secured to an end of the drive cable, and a cable retainment feature provided on the cable pulley and operable to retain the termination crimp within the crimp pocket once received therein, wherein the termination crimp is received within the crimp pocket by forcing the termination crimp past the cable retainment feature.

Each of embodiments A and B may have one or more of the following additional elements in any combination: Element 1: wherein the termination crimp is selected from the group consisting of a ball crimp, a barrel crimp, a puck-shaped crimp, a truncated cone, a rectangular prism, a ball and shank, a barrel with flange, and any combination thereof. Element 2: wherein the capstan further provides upper and lower pulley flanges that cooperatively define the pulley groove, and a termination aperture defined by the cable pulley and contiguous with the crimp pocket, the termination aperture being sized to receive and guide the termination crimp into the crimp pocket. Element 3: wherein at least one of the termination aperture and the crimp pocket is cooperatively defined by the upper and lower flanges. Element 4: wherein the cable retainment feature comprises one or more retention bumps located within the termination aperture. Element 5: wherein the one or more retention bumps comprise a first retention bump provided on the upper pulley flange and a second retention bump provided on the lower pulley flange and vertically offset from the first retention bump. Element 6: wherein the crimp pocket comprises an arcuate cavity providing first and second pocket chambers separated by the one or more retention bumps. Element 7: wherein the cable retainment feature is located within the crimp pocket and comprises a pair of retention bumps angularly separated by an arcuate surface. Element 8: wherein the crimp pocket comprises an arcuate cavity providing first and second pocket chambers separated by the cable retainment feature, and wherein the termination crimp enters a given one of the first or second pocket chambers by bypassing an adjacent retention bump of the pair of retention bumps.

Element 9: wherein the capstan further provides upper and lower pulley flanges that cooperatively define the pulley groove, and a termination aperture defined by the cable pulley and contiguous with the crimp pocket, the termination aperture being sized to receive and guide the termination crimp into the crimp pocket. Element 10: wherein at least one of the termination aperture and the crimp pocket is cooperatively defined by the upper and lower flanges. Element 11: wherein the cable retainment feature comprises one or more retention bumps located within the termination aperture, and wherein each retention bump comprises an elongate protrusion extending substantially perpendicular to a rotational axis of the capstan and further extending transversely through the crimp pocket. Element 12: wherein the one or more retention bumps comprise a first retention bump provided on the upper pulley flange and a second retention bump provided on the lower pulley flange and vertically offset from the first retention bump. Element 13: wherein the crimp pocket comprises an arcuate cavity providing first and second pocket chambers separated by the one or more retention bumps. Element 14: wherein each pocket chamber defines a pocket window through which a portion of the termination crimp is visible when the termination crimp is received therein. Element 15: wherein the cable retainment feature is located within the crimp pocket and comprises a pair of retention bumps angularly separated by an arcuate surface. Element 16: wherein the crimp pocket comprises an arcuate cavity providing first and second pocket chambers separated by the cable retainment feature, and wherein the termination crimp enters a given one of the first or second pocket chambers by bypassing an adjacent retention bump of the pair of retention bumps. Element 17: wherein the capstan comprises first and second component parts that are joined to form the capstan, and wherein joining the first and second component parts forms the cable pulley and the crimp pocket. Element 18: wherein joining the first and second component parts secures the termination crimp within the crimp pocket. Element 19: wherein the capstan comprises a driven capstan and the capstan assembly further includes a drive capstan including a drive gear and extending from or forming part of the drive input such that rotation of the drive input correspondingly rotates the drive gear, and a driven gear provided on the drive capstan and positioned to intermesh with the drive gear such that rotating the drive gear correspondingly rotates the driven gear and the cable pulley.

By way of non-limiting example, exemplary combinations applicable to A and B include: Element 2 with Element 3; Element 3 with Element 4; Element 4 with Element 4; Element 4 with Element 6; Element 2 with Element 7; Element 7 with Element 8; Element 9 with Element 10; Element 9 with Element 11; Element 11 with Element 12; Element 11 with Element 13; Element 13 with Element 14; Element 9 with Element 15; Element 15 with Element 16; and Element 17 with Element 18.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A surgical tool, comprising:
a drive housing;
a drive input rotatably mounted to a bottom of the drive housing; and
a capstan assembly arranged within the drive housing and operatively coupled to the drive input such that rotation of the drive input correspondingly actuates the capstan assembly, the capstan assembly including a capstan that provides:
a cable pulley with a pulley groove sized to receive a drive cable wrapped at least partially about the capstan;
a crimp pocket defined in the cable pulley and sized to receive a termination crimp secured to an end of the drive cable; and
a cable retainment feature provided on the cable pulley and operable to retain the termination crimp within the crimp pocket once received therein,
wherein the termination crimp is received within the crimp pocket by forcing the termination crimp past the cable retainment feature.

2. The surgical tool of claim 1, wherein the termination crimp is selected from the group consisting of a ball crimp, a barrel crimp, a puck-shaped crimp, a truncated cone, a rectangular prism, a ball and shank, a barrel with flange, and any combination thereof.

3. The surgical tool of claim 1, wherein the capstan further provides:
upper and lower pulley flanges that cooperatively define the pulley groove; and
a termination aperture defined by the cable pulley and contiguous with the crimp pocket, the termination aperture being sized to receive and guide the termination crimp into the crimp pocket.

4. The surgical tool of claim 3, wherein at least one of the termination aperture and the crimp pocket is cooperatively defined by the upper and lower flanges.

5. The surgical tool of claim 3, wherein the cable retainment feature comprises one or more retention bumps located within the termination aperture.

6. The surgical tool of claim 5, wherein the one or more retention bumps comprise a first retention bump provided on the upper pulley flange and a second retention bump provided on the lower pulley flange and vertically offset from the first retention bump.

7. The surgical tool of claim 5, wherein the crimp pocket comprises an arcuate cavity providing first and second pocket chambers separated by the one or more retention bumps.

8. The surgical tool of claim 3, wherein the cable retainment feature is located within the crimp pocket and comprises a pair of retention bumps angularly separated by an arcuate surface.

9. The surgical tool of claim 8, wherein the crimp pocket comprises an arcuate cavity providing first and second pocket chambers separated by the cable retainment feature, and wherein the termination crimp enters a given one of the first or second pocket chambers by bypassing an adjacent retention bump of the pair of retention bumps.

10. A capstan assembly for a surgical tool, comprising:
a capstan arrangeable within a drive housing of the surgical tool and operatively couplable to a drive input rotatably mounted to a bottom of the drive housing such that rotation of the drive input correspondingly actuates the capstan, the capstan including:
a cable pulley with a pulley groove sized to receive a drive cable wrapped at least partially about the capstan;
a crimp pocket defined in the cable pulley and sized to receive a termination crimp secured to an end of the drive cable; and
a cable retainment feature provided on the cable pulley and operable to retain the termination crimp within the crimp pocket once received therein,
wherein the termination crimp is received within the crimp pocket by forcing the termination crimp past the cable retainment feature.

11. The capstan assembly of claim 10, wherein the capstan further provides:
upper and lower pulley flanges that cooperatively define the pulley groove; and
a termination aperture defined by the cable pulley and contiguous with the crimp pocket, the termination aperture being sized to receive and guide the termination crimp into the crimp pocket.

12. The capstan assembly of claim 11, wherein at least one of the termination aperture and the crimp pocket is cooperatively defined by the upper and lower flanges.

13. The capstan assembly of claim 11, wherein the cable retainment feature comprises one or more retention bumps located within the termination aperture, and wherein each retention bump comprises an elongate protrusion extending substantially perpendicular to a rotational axis of the capstan and further extending transversely through the crimp pocket.

14. The capstan assembly of claim 13, wherein the one or more retention bumps comprise a first retention bump provided on the upper pulley flange and a second retention bump provided on the lower pulley flange and vertically offset from the first retention bump.

15. The capstan assembly of claim 13, wherein the crimp pocket comprises an arcuate cavity providing first and second pocket chambers separated by the one or more retention bumps.

16. The capstan assembly of claim 15, wherein each pocket chamber defines a pocket window through which a portion of the termination crimp is visible when the termination crimp is received therein.

17. The capstan assembly of claim 11, wherein the cable retainment feature is located within the crimp pocket and comprises a pair of retention bumps angularly separated by an arcuate surface.

18. The capstan assembly of claim 17, wherein the crimp pocket comprises an arcuate cavity providing first and second pocket chambers separated by the cable retainment feature, and wherein the termination crimp enters a given one of the first or second pocket chambers by bypassing an adjacent retention bump of the pair of retention bumps.

19. The capstan assembly of claim 10, wherein the capstan comprises first and second component parts that are joined to form the capstan, and wherein joining the first and second component parts forms the cable pulley and the crimp pocket.

20. The capstan assembly of claim 19, wherein joining the first and second component parts secures the termination crimp within the crimp pocket.

21. The capstan assembly of claim 10, wherein the capstan comprises a driven capstan and the capstan assembly further includes:
a drive capstan including a drive gear and extending from or forming part of the drive input such that rotation of the drive input correspondingly rotates the drive gear; and
a driven gear provided on the drive capstan and positioned to intermesh with the drive gear such that rotating the drive gear correspondingly rotates the driven gear and the cable pulley.
